(19) United States
(12) Patent Application Publication
(10) Pub. No.: US 2004/0234437 A1
(43) Pub. Date: Nov. 25, 2004

(54) Treatment of materials

(75) Inventor: Alan Colin McGowan, Godalming (GB)

Correspondence Address: LAHIVE & COCKFIELD, LLP., 28 STATE STREET, BOSTON, MA, 02109, US

(73) Assignee: Flute Developments Limited, Godalming (GB)

(21) Appl. No.: 10/746,892

(22) Filed: Dec. 23, 2003

(30) Foreign Application Priority Data
Dec. 24, 2002 (GB) 0229986.5

(51) Int. Cl. A61L 2/00

(52) U.S. Cl. 422/295; 422/292; 422/39

(57) ABSTRACT

Apparatus for the treatment of material comprises a confined volume for receiving the material, and a source of gas at elevated pressure communicable with the interior of the volume. A suitable source of gas is an airgun, or other apparatus capable of releasing a volume of gas at elevated pressure such as at least 1000 psi. It is preferred that the source of gas is located within the confined volume. It is also preferred that the confined volume is a pressure vessel. Inlet an outlet valves will assist in loading and unloading material for treatment. Gate valves will further assist by closing when the volume is full. The invention is thus particularly useful in the destruction of chemical and biological agents, for the purification of contaminated water, and for the destruction of tropical and other larvae previously leading to diseases such as Nile Disease, malaria and the like.

### FIELD OF THE INVENTION

The present application provides a means for the treatment of material. It disrupts complex molecular structures and thereby reduces certain molecules to inert compounds.

### BACKGROUND ART

The invention is thus particularly (but not exclusively) useful in the destruction of chemical and biological agents, for the purification of contaminated water, and for the destruction of tropical and other larvae previously leading to diseases such as Nile Disease, malaria and the like.

### SUMMARY OF THE INVENTION

Apparatus for the treatment of material, comprising a confined volume for receiving the material, and a source of gas at elevated pressure communicable with the interior of the volume.

A suitable source of gas is an airgun, or other apparatus capable of releasing a volume of gas at elevated pressure such as at least 1000 psi.

It is preferred that the source of gas is located within the confined volume.

It is also preferred that the confined volume is a pressure vessel.

Inlet an outlet valves will assist in loading and unloading material for treatment. Gate valves will further assist by closing when the volume is full.

Other preferred features of the present invention will be apparent from the non-limiting description set forth below.

### BRIEF DESCRIPTION OF THE DRAWING

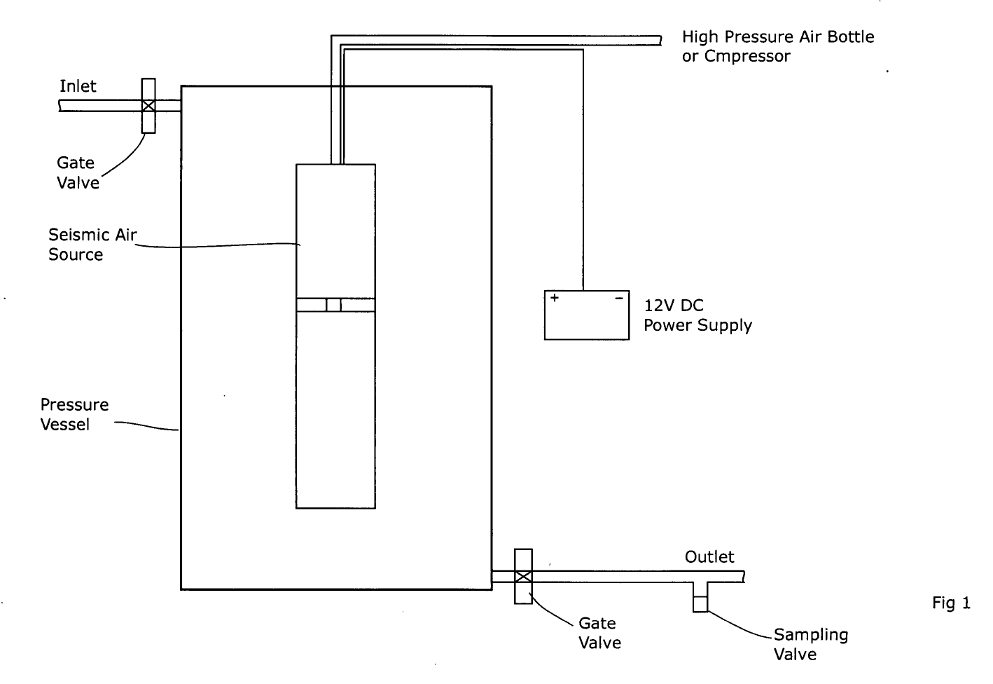

An embodiment of the present invention will now be described by way of example, with reference to the accompanying FIG. 1, which is a schematic illustration of the apparatus.

### DETAILED DESCRIPTION OF THE EMBODIMENT

The apparatus is constructed around a pressure vessel, suitably cylindrical in shape, and of a size to suit the required volume of output. A vessel is a minimum of 3″ in thickness will be able to withstand the typical pressure involved in use. It will ideally be constructed of stainless steel. Situated at the top of the vessel is an inlet valve, which can be either automatically operated by an electronic sensor switch, or by manual control. In this embodiment, the inlet is a gate valve, as will be explained later.

At the bottom of the vessel is an outlet valve, in this case a similar gate valve operated either electrically or by manual means. There is also provision for a sampling valve, in order to draw off by-products for testing. As shown in FIG. 1, the sampling valve is present on the outlet line but could if desired be arranged to tap material from the pressure vessel. In this way, material could be checked to see if it needed further treatment.

Situated within the pressure vessel, there is a seismic air source. In this embodiment the source is an air-gun of the type that is commercially available for different purposes. This can be of varied volume capacity, depending upon the throughput required by the user. Suitable capacities are pressure chambers of 10, 20, 40, or 100 cubic inches. As the required volume increases, so does the size of the pressure vessel. Air guns and their construction are described more fully in my earlier patent application WO98/10169.

Material enters the vessel via the inlet valve, until the valve is closed manually or the material reaches such a level that the flow is cut off by the gate valve. The material should be in a fluid form or should have been rendered into such a form. The seismic air source or air-gun is charged with high pressure air, from 0 to 2000 psi, either by a static compressor or by a bank of high pressure air bottles.

The seismic air source or air-gun is then fired. In some airguns, this is via by an electrical charge of (typically) 12 volts dc. This causes an expanding air bubble of immense pressure which, upon reaching the extent of expansion (i.e. the wall of the pressure, vessel) implodes upon itself and, in turn, destroys the molecular structure of the material being treated.

A bleed off or sampling valve enables samples to be drawn off for analysis, and, if necessary, adjustment of the air pressure, enabling the operator to achieve the destruction of whatever chemical, biological or other agent, is being treated at the time.

1. Apparatus for the treatment of material, comprising a confined volume for receiving the material, and a source of gas at elevated pressure communicable with the interior of the volume.

2. Apparatus according to claim 1 in which the source of gas is an airgun.

3. Apparatus according to claim 1 in which the source of gas is adapted to release a volume of gas at a pressure of at least 1000 psi.

4. Apparatus according to claim 1 in which the source of gas is located within the confined volume.

5. Apparatus according to claim 1 in which the confined volume is a pressure vessel.

6. Apparatus according to claim 1 in which the confined volume is provided with at least one of an inlet and an outlet valve.

7. Apparatus according to claim 1 in which the confined volume is provided with inlet valve in the form of a gate valve.
